(19) United States
(12) Patent Application Publication
(10) Pub. No.: US 2009/0076583 A1
(43) Pub. Date: Mar. 19, 2009

(54) MEDICAL ELECTRODE

(75) Inventors: Olaf Schermeier, Lubeck (DE); Thomas Grassl, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: Drager Medical AG & Co. KG, Lubeck (DE)

(21) Appl. No.: 12/209,352

(22) Filed: Sep. 12, 2008

(30) Foreign Application Priority Data
Sep. 14, 2007 (DE) 10 2007 044 020.2

(51) Int. Cl. A61N 1/04

(52) U.S. Cl. 607/149

(57) ABSTRACT

A medical electrode is provided for detecting and transmitting electrical pulses from the body surface of a patient to a device for processing the electrical pulses. The medical electrode includes a contact element (1) with a conductive material (4) for passing on the electrical pulses to the device processing the electrical pulses and with a conductive area (3) for establishing contact with the body surface of the patient. A first adhesion area (6) is provided adjoining the conductive area (3) for positioning the contact element (1) on the body surface of the patient. An attaching element (2) is provided with a second adhesion area (7) formed on the underside of the attaching element (2) for attaching the contact element (1) on the body surface of the patient. Said attaching element is arranged concentrically in relation to said contact element.

### CROSS REFERENCE TO RELATED APPLICATIONS

This application claims the benefit of priority under 35 U.S.C. §119 of German Patent Application DE 10 2007 044 020.2 filed Sep. 14, 2007, the entire contents of which are incorporated herein by reference.

### FIELD OF THE INVENTION

The present invention pertains to a medical electrode for detecting and transmitting electrical pulses from the body surface of a patient to a means processing the electrical pulses.

### BACKGROUND OF THE INVENTION

Medical electrodes, especially disposable electrodes, for detecting and transmitting electrical pulses from the body surface of a patient to a means processing the electrical pulses are known from practice. The lead-off of electrical pulses from the body surface of a patient depends on both the position of the electrode and a good electrical contact between the electrode and the body surface of the patient. To obtain a good electrical contact, the patient's skin is usually cleaned beforehand with alcohol in order to remove dirt and skin fat. However, it may become necessary for the user despite optimal preparation of the patient's skin to reposition the medical electrode on the body surface of the patient because of an excessively low signal intensity of the electrical pulses. A plurality of repositioning of the medical electrodes are at times necessary in medical practice until the electrical pulses are obtained with the necessary signal intensity. The electrode, once applied, must be removed again in order to be reattached in another position. Each new position of the electrode causes a reduction of adhesiveness. Reduced adhesiveness of the medical electrodes may lead to an unintended detachment during a medical procedure and thus to an interruption in the monitoring of the means processing the electrical pulses. The patients are covered, as a rule, under sterile conditions during a surgery, as a result of which replacement of medical electrodes is made additionally difficult or prevented.

To prevent the detachment of an electrode already repositioned several times, a new medical electrode, which is placed in the final position of the body surface of the patient, is therefore frequently used in medical practice. However, the use of a new medical electrode causes additional costs and additional waste.

### SUMMARY OF THE INVENTION

Based on the state of the art, the object of the present invention is to provide a medical electrode, which makes possible simple and cost-effective handling during the preparation for readiness to operate.

Thus, the present invention proposes a medical electrode for detecting and transmitting electrical pulses from the body surface of a patient to a means processing the electrical pulses, which means comprises a conductive material for passing on the electrical pulses to the means processing the electrical pulses, wherein the contact element has a conductive area for establishing contact with the body surface of the patient and a first adhesion area adjoining the conductive area for positioning the contact element on the body surface of the patient. The medical electrode according to the present invention comprises, furthermore, an attaching element with a second adhesion area formed on the underside of the attaching element for attaching the contact element to the body surface of the patient. The attaching element is arranged concentrically in relation to said contact element. This means that in the search for an optimal electrode position, the medical electrode according to the present invention can be attached in a simple manner in a first step by connecting the first adhesion area to the body surface of the patient and can be removed again before the medical electrode is fixed in another step in the final position by connecting the second adhesion area to the body surface of the patient.

The first adhesion area is advantageously made with a smaller area compared to the second adhesion area. The contact element can thus be easily detached from the body surface of the patient in a simple manner for a necessary repositioning and fixed in a stable manner for a final positioning. To prevent premature adhesion of the second adhesion area, the second adhesion area advantageously has a protective cover, which can be removed before use and is detached only before the final positioning of the electrode.

In another advantageous embodiment of the medical electrode according to the present invention, the contact element and the substrate may be of one part each. In the search for the optimal position on the body surface of the patient, only the contact element is advantageously positioned at first in this embodiment before the attaching element is connected to the contact element in the final position of the medical electrode according to the present invention in such a way that the contact element is fixed correspondingly on the body surface by connecting the second adhesion area to the body surface of the patient.

In yet another preferred embodiment, the attaching element of the medical electrode according to the present invention is perforated at a defined number of geometrically distributed dots, and at least some of the dots form an outer edge with at least one inner attaching element and the dots are perforated such that the at least one inner attaching element is detachable from the attaching element. At least two medical electrodes of different size can thus be advantageously made available to the user, and the user can prepare the particular necessary electrode size in a simple manner and rapidly. One advantage of this preferred embodiment is, moreover, that only one product must be kept stocked in the clinical practice for use on at least two patients of different size.

Furthermore, the second adhesion area formed on the attaching element may have openings. The openings make possible better handling of the medical electrode according to the present invention during the final positioning. The second adhesion area of the attaching element may be divided into separate adhesion areas in another preferred embodiment. Every individual adhesion area may have a separate protective film in this embodiment. The individual adhesion areas may be connected separately to the body surface of the patient.

The present invention will be explained in more detail with reference to the attached drawings, the same reference numbers designating identical structures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic sectional view of a medical electrode according to the state of the art;

FIG. 2 is a schematic sectional view of a first embodiment of a medical electrode according to the present invention;

FIG. 3A is a schematic sectional exploded view of a second embodiment of a medical electrode according to the present invention, showing each of the contact element and the attaching element as a one-part design;

FIG. 3B is a schematic sectional view of the second embodiment of a medical electrode according to the present invention, showing the contact element connected to the attaching element;

FIG. 4 is a schematic sectional view of the second adhesion area of the medical electrode according to the present invention in an embodiment with an opening;

FIG. 5 is a schematic sectional view of the second adhesion area of the medical electrode according to the present invention in an embodiment with a plurality of openings; and

FIG. 6 is a medical electrode according to the present invention in an embodiment with two different sizes.

### DESCRIPTION OF THE PREFERRED EMBODIMENTS

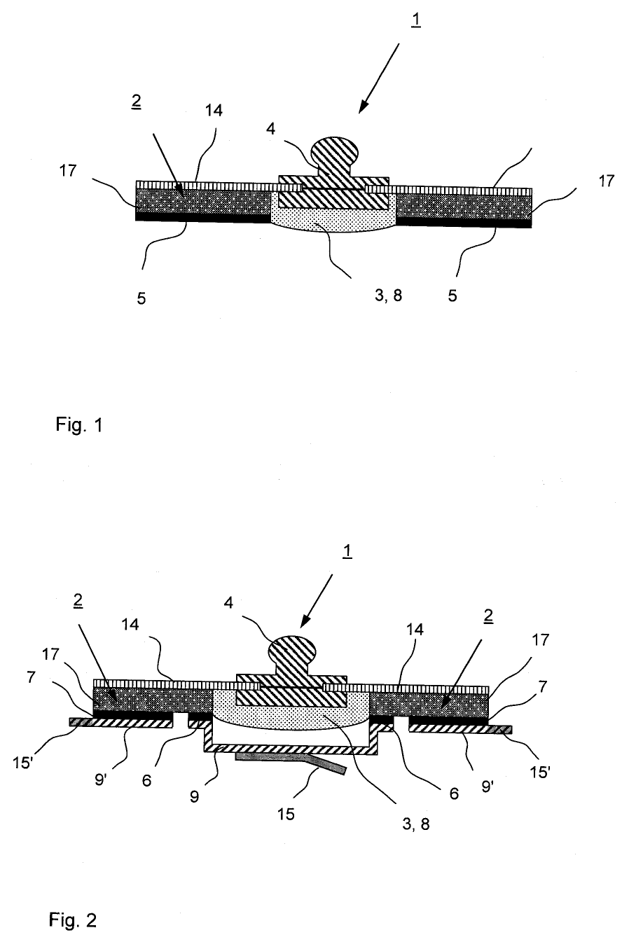

Referring to the drawings in particular, FIG. 1 shows a medical electrode according to the state of the art. A contact element 1 is intrinsically tied to an attaching element 2 as in a base layer 14. The attaching element 2 fixes the contact element 1 on the body surface of a patient. The attaching element 2 has an adhesion area 5, which is formed on the underside of a carrier 17 and attaches the contact element 1 on the skin surface of the patient. A conductive area 3, which is enriched especially with a hydrogel 8, is used to detect electric pulses from the body surface of a patient. The electrical pulses are passed on via a conductive material 4 of the contact element 1 to a means (not shown) processing the electrical pulses.

The schematic sectional view in FIG. 2 shows a first embodiment of the medical electrode according to the present invention. A contact element 1 comprises a conductive material 4 for passing on electrical pulses to a means (not shown) processing electrical pulses, a conductive area 3 for establishing contact with the body surface of the patient, and a first adhesion area 6 adjoining the conductive area 3 for positioning the contact element 1 on the body surface of the patient. The conductive area 3 is enriched with a layer of hydrogel 8. The electrical pulses are detected through the conductive area 3 shown in the view in FIG. 2 from the body surface of the patient and passed on via the conductive material 4 and a cable to the means (not shown) processing electrical pulses. The conductive material 4 may consist of steel and silver chloride. The conductive material 4 is made of carbon in an especially preferred embodiment. The cable for connection to the means processing the electrical pulses can be connected to the contact element 1 via a welded connection.

Furthermore, the medical electrode according to the present invention comprises an attaching element 2 with a second adhesion area 7 formed on the underside of the attaching element 2 for attaching the contact element 1 on the body surface of the patient. The contact element 1 is arranged in the center of the medical electrode according to the present invention. The attaching element 2 is arranged concentrically in relation to the contact element 1. The attaching element 2 is connected by a carrier 17 and preferably by a base layer 14 to the contact element 1, on the underside of which the first adhesion area 6 and at a radially spaced location from which the second adhesion area 7 are formed. The carrier 17 advantageously consists of a foamed or textile material, for example, plaster material. The first adhesion area 6 and the second adhesion area 7 are arranged at spaced locations from one another in space at the lower surface of the carrier 17. At least the second adhesion area 7 is covered with a protective cover 9′. The side of the attaching element 2 facing away from the body surface may be provided with a cover layer 14, and the cover layer 14 is connected to the contact element 1.

Due to this design according to the present invention, the medical electrode can be advantageously positioned in the search for an optimal position for leading electrical pulses from the body surface of the patient on the body surface in a first step and finally attached on the body surface of said patient in a second step when a sufficiently high signal intensity of the electrical pulses is present. The first adhesion area 6 is needed during the phase of positioning, whereas the second adhesion area 7 is used only during the phase of fixation. Both the first adhesion area 6 and the second adhesion area 7 have a protective cover 9 and 9′ each for this, the protective cover 9 being provided with a gripping area 15. The gripping area 15 makes possible the rapid exposure of the first adhesion area 6. The protective cover 9′ may be extended towards the attaching element 2 on at least one side, as a result of which this extension forms a gripping area 15′.

For simple repositioning and optimal fixation of the contact element 1, the first adhesion area 6 comprises a smaller area compared to the second adhesion area 7. The contact element 1 can thus be removed again from the body surface of the patient in a simple manner by detaching the first adhesion area 6 from the body surface of the patient and placed on another side of the body surface of the patient. The contact element 1 can be fixed in a stable form in a final position by connecting the second adhesion area 7 to the body surface of the patient.

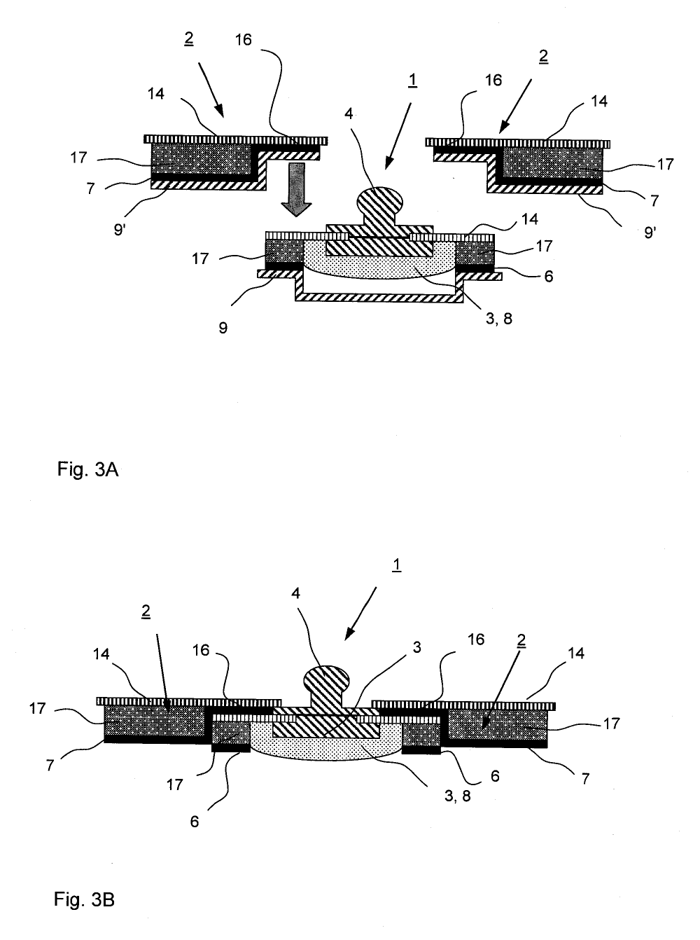

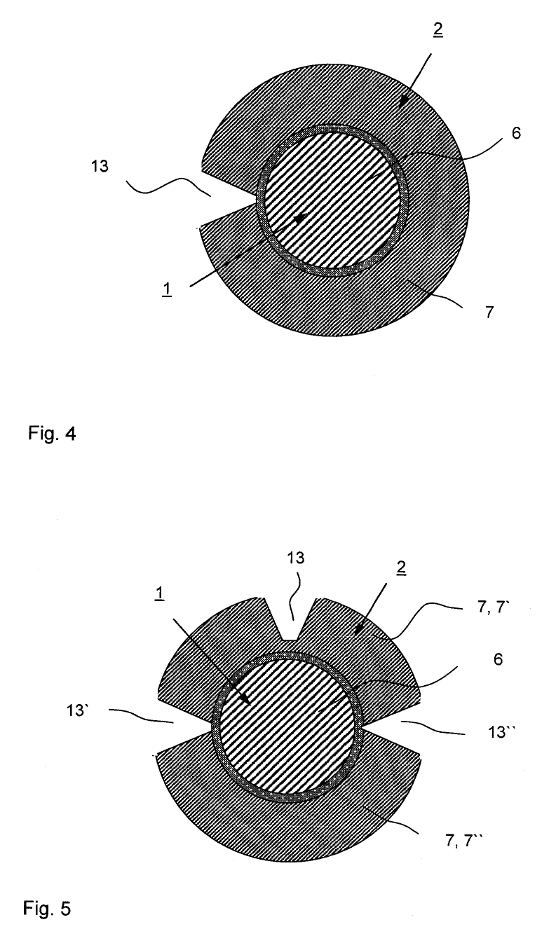

The schematic sectional view in FIG. 3A and FIG. 3B shows a second embodiment of the medical electrode according to the present invention. The contact element 1 and the attaching element 2 are each of a one-part design. Both the contact element 1 and the attaching element 2 preferably have a circular base. In this embodiment, the contact element 1 has a carrier 17, which is arranged concentrically around the conductive material 4 and preferably consists of a foamed or textile material. The conductive area 3 is enriched with a layer of hydrogel, just as in the embodiment according to FIG. 2. The first adhesion area 6 is formed on the underside of the carrier 17. The carrier 17 as well as a base 14 stabilize the contact element 1 especially the layer of hydrogel 8 of the conductive area 3. Both the conductive area 3, especially the layer of hydrogel 8, and the first adhesion area 6 are covered with a protective cover 9. The protective cover 9 may be provided, just as in the embodiment according to FIG. 2, with a gripping area (not shown). The attaching element 2 comprises a carrier 17 and a base layer 14. The base layer 14 is preferably made of a thin film. The lower surface of the base layer 14 is divided in two. A first surface forms a connection area 16, while a second surface is connected to the carrier 17. The connection area 16 connects the attaching element 2 to the top side of the contact element 1. The lower surface of the carrier 17 is designed as the second adhesion area 7.

The search for an optimal position for leading electrical pulses from the body surface of the patient is performed during the phase of positioning with the contact element 1. If a final lead position has been found, the contact element 1 is fixed in this position by connecting, in a first step, at least a part of the surface of the contact element 1 to the connection area 16 of the attaching element 2 and by placing the second adhesion area 7 on the body surface of the patient in a second step during the phase of fixation after removing the protective cover 9′. In another embodiment, both the connection area 16 and the second adhesion area 7 can be covered with a protective cover each or with a common protective cover (not shown). A medical electrode fixed on the surface of a patient is shown in FIG. 3B.

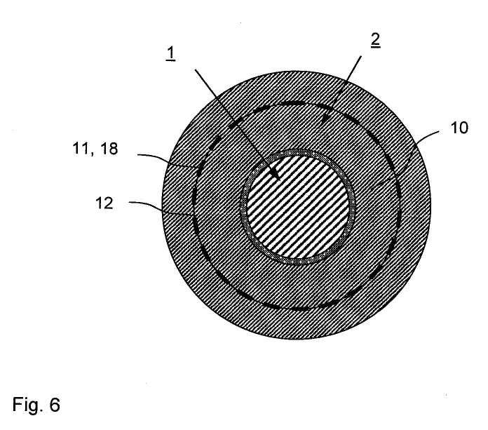

The sectional view in FIG. 4 shows the second adhesion area 7 of the attaching element 2 in an embodiment variant with a lateral opening 13. The opening 13 makes possible the simple handling of the attaching element 2, for example, at the time of the final fixation of the contact element 1 on the body surface of the patient. The sectional view in FIG. 5 shows an embodiment of the second adhesion area 7 with a plurality of openings 13, 13′, 13″ which divide the second adhesion area 7 into separate adhesion areas 7′ and 7″. The separate second adhesion areas 7′ and 7″ can be covered each with individual protective covers.

The sectional view in FIG. 6 shows another embodiment variant of the attaching element 2 of the medical electrode according to the present invention. The attaching element 2 is perforated in a defined number of geometrically distributed dots 12, and at least some of the dots 12 form an outer edge 11 of at least one inner attaching element 10 and the dots 12 are perforated such that at least one inner attaching element 10 is detachable from the attaching element 2. The medical electrode according to the present invention can thus be prepared rapidly and in a simple manner with a corresponding size, according to the size of the patient, before it is put into operation. The inner attaching element 10 is separated from the attaching element 2 for this. The inner attaching element 10 is advantageously made with a separation area 18, which is limited by a part of the outer edge 11 of the inner attaching element 10. The edge area of the separation area 18, which agrees to a part of the outer edge 11 of the inner attaching element 10, is preferably perforated continuously. This makes it possible to separate the inner attaching element 10 from the attaching element 2 in a simple manner. The inner attaching element 10 forms a part of the base of attaching element 2.

The medical electrode according to the present invention is used with the complete area of the attaching element 2 for a large patient, whereas the medical electrode according to the present invention is used with the base of the inner attaching element 10 for smaller patients, for example, children.

While the present invention has been described with reference to the preferred exemplary embodiments, various changes and modifications are clear to the person skilled in the art. All these changes and modifications shall fall within the scope of protection of the claims attached. While specific embodiments of the invention have been shown and described in detail to illustrate the application of the principles of the invention, it will be understood that the invention may be embodied otherwise without departing from such principles.

### APPENDIX

### LIST OF REFERENCE NUMBERS

1 Contact element
2 Attaching element
3 Conductive area
4 Conductive material
5 Adhesion area
6 First adhesion area
7, 7′, 7″ Second adhesion area
8 Hydrogel
9, 9′ Protective cover
10 Inner attaching element
11 Outer edge of inner attaching element
12 Geometrically distributed dots
13 Opening
14 Base layer
15, 15′ Gripping area
16 Connection area
17 Carrier
18 Separation area

What is claimed is:

1. A medical electrode for detecting and transmitting electrical pulses from the body surface of a patient to a device for processing the electrical pulses, the medical electrode comprising:
a contact element with a conductive area for establishing contact with the body surface of the patient;
a first adhesion area adjoining said conductive area for positioning said contact element on the body surface of the patient; and
an attaching element with a second adhesion area formed on the underside of said attaching element for attaching said contact element on the body surface of the patient said attaching element being arranged concentrically in relation to said contact element.

2. A medical electrode in accordance with claim 1, wherein said first adhesion area is made with a smaller attachment surface area than an attachment surface area of said second adhesion area.

3. A medical electrode in accordance with claim 1, wherein said contact element and said attaching element are each of a one-part design.

4. A medical electrode in accordance with claim 1, wherein at least one of said attaching element and said contact element has a substrate, said substrate being made of one of a foamed material and a textile material.

5. A medical electrode in accordance with claim 1, wherein said second adhesion area has a removable protective cover in contact with an attachment surface area of said second adhesion area, said protective cover being removed from said attachment surface area of said second adhesion area before use.

6. A medical electrode in accordance with claim 5, wherein said protective cover has a gripping area for removing said protective cover.

7. A medical electrode in accordance with claim 1, wherein said second adhesion area has one or more openings.

8. A medical electrode in accordance with claim 7, wherein at least two said openings divide said second adhesion area into plural separate attachment surface areas.

9. A medical electrode in accordance with claim 1, wherein said attaching element comprises a defined number of said geometrically distributed dots, wherein at least some of said dots form an outer edge of an inner attaching element and said at least some of said dots are perforated such that said inner attaching element is detachable from a remainder of said attaching element.

10. A medical electrode for detecting and transmitting electrical pulses from the body surface of a patient to a device for processing the electrical pulses, the medical electrode comprising:
a contact element with a conductive contact area for establishing contact with the body surface of the patient;
a first adhesion area adjoining said conductive area for positioning said contact element on the body surface of the patient; and
an attaching element connected to said contact element, said attaching element having a second adhesion area for attaching said contact element on the body surface of the patient said attaching element being arranged concentrically in relation to said contact element.

11. A medical electrode in accordance with claim 10, wherein said first adhesion area is made with a smaller attachment surface area than an attachment surface area of said second adhesion area.

12. A medical electrode in accordance with claim 10, wherein said contact element and said attaching element are each of a one-part design.

13. A medical electrode in accordance with claim 10, wherein at least one of said attaching element and said contact element has a substrate, said substrate being made of one of a foamed material and a textile material.

14. A medical electrode in accordance with claim 10, wherein said second adhesion area has a removable protective cover in contact with an attachment surface area of said second adhesion area, said protective cover being removed from said attachment surface area of said second adhesion area before use.

15. A medical electrode in accordance with claim 14, wherein said protective cover has a gripping area for removing said protective cover.

16. A medical electrode in accordance with claim 10, wherein said second adhesion area has one or more interruptions.

17. A medical electrode in accordance with claim 16, wherein at least two said interruption divide said second adhesion area into plural separate attachment surface areas.

18. A medical electrode in accordance with claim 10, wherein said attaching element comprises a defined number of perforations to define an inner attaching element, said inner attaching element is detachable from a remainder of said attaching element.
